(19) United States
(12) Patent Application Publication
(10) Pub. No.: US 2003/0113386 A1
(43) Pub. Date: Jun. 19, 2003

(54) Composition, particularly cosmetic, combining DHEA and/or a precursor or derivative thereof, and at least an agent for increasing glycosaminoglycan synthesis

(75) Inventors: Josiane Allec, Antibes (FR); Nathalie Cleren, Rueil Malmaison (FR)

Correspondence Address: OBLON, SPIVAK, MCCLELLAND, MAIER & NEUSTADT, P.C., FOURTH FLOOR, 1755 JEFFERSON DAVIS HIGHWAY, ARLINGTON, VA, 22202, US

(73) Assignee: L'OREAL, Paris (FR)

(21) Appl. No.: 10/340,662

(22) Filed: Jan. 13, 2003

Related U.S. Application Data
(63) Continuation of application No. 09/903,766, filed on Jul. 13, 2001, now abandoned.

(30) Foreign Application Priority Data
Jul. 13, 2000 (FR) 0009221

(51) Int. Cl. A61K 35/78; A61K 35/84

(52) U.S. Cl. 424/725; 424/195.16; 424/401

(57) ABSTRACT

A composition, comprising, in a physiologically acceptable medium, DHEA and/or a chemical or biological precursor or derivative thereof, in combination with at least one compound which increases glycosaminoglycan synthesis, or a natural extract containing the compound.

### BACKGROUND OF THE INVENTION

1. Field of the Invention

The present invention relates to a composition comprising, in a physiologically acceptable medium, DHEA and/or a chemical or biological precursor or derivative thereof, in combination with at least one compound which increases glycosaminoglycan synthesis, or a natural extract containing it, and also to its cosmetic uses.

2. Description of the Background

It is known that the signs of ageing of the skin arise not only from chronological causes (genetically programmed ageing), but also are caused by additional aggravating factors such as exposure to UV radiation and especially hormonal deficiencies arising during menopause. These factors are reflected by changes in the condition of the skin which are generally observed by women and confirmed by clinical studies, namely:

a loss of firmness, resilience, elasticity and volume of the skin, and, at a more advanced age, a papery appearance of the skin,

dryness of the skin with loss of suppleness, appearance of squama and rough patches,

a loss of radiance of the complexion, which becomes dull, and appearance of brown marks.

These changes are essentially attributable to a slowing-down and imbalance in cutaneous functioning, which is reflected by an “atrophy” of all of the strata of the skin. Thus, a reduction in the quality of the dermis (elastin, collagen, glycosaminoglycans) and a loss of consistency of the extracellular matrix; a reduction in the thickness of the epidermis (slowing down of cell renewal); a slowing-down of the production of epidermal structural proteins and lipids; an imbalance in desquamation; and a reduction in the water content of the skin, are observed.

Means for countering, or at the very least for slowing down, the appearance of these signs of ageing and thus for looking younger for a longer time, are constantly being sought.

The Applicants have now discovered that quality of the skin can be preserved and improved by combining DHEA and/or precursors or derivatives thereof with an agent for increasing glycosaminoglycan synthesis.

DHEA, or dehydroepiandrosterone, is a natural steroid produced essentially by the adrenocortical glands. It is known for its capacity to promote epidermal keratinization (JP-07 196 467) and for its treatment of dry skin, because of its ability to increase the endogenous production and secretion of sebum and to strengthen the skin's barrier effect (U.S. Pat. No. 4,496,556). U.S. Pat. No. 5,843,932 also discloses the use of DHEA to remedy dermal atrophy by inhibiting the loss of collagen and connective tissue. Finally, it has been proposed to use DHEA sulfate to treat various signs of ageing such as wrinkles, loss of radiance of the skin and slackening of the skin (EP-0 723 775). However, to the Applicants' knowledge, there is no suggestion in the art to combine DHEA and/or precursors or derivatives thereof with a compound which increases glycosaminoglycan synthesis.

### SUMMARY OF THE INVENTION

Accordingly, one object of the present invention is to provide a formulation which enhances the activity of dehydroepiandrosterone on the skin.

Briefly, this object and other objects of the invention as hereinafter will become more readily apparent can be attained by a composition comprising, in a physiologically acceptable medium, DHEA and/or a chemical or biological precursor or derivative thereof, in combination with at least one compound which increases glycosaminoglycan synthesis.

### DETAILED DESCRIPTION OF THE PREFERRED EMBODIMENTS

DHEA has the formula (I) below:

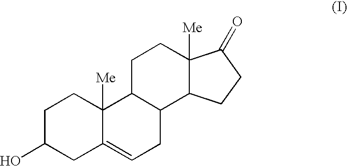

DHEA is available, for example, from the company Akzo Nobel.

The expression “DHEA precursors” means the biological precursors thereof which may be converted into DHEA during metabolism, and also chemical precursors thereof which may be converted into DHEA by exogenous chemical reaction. Examples of biological precursors are Δ5-pregnenolone, 17α-hydroxypregnenolone and 17α-hydroxypregnenolone sulfate, although this list is not limiting. Examples of chemical precursors are sapogenins and derivatives thereof, such as diosgenin (or spirost-5-en-3-β-ol), hecogenin, hecogenin acetate, smilagenin and sarsapogenin, and also plant extracts containing them, in particular fenugreek and Dioscorea extracts such as the root of wild yam, although this list is not limiting.

According to another preferred embodiment, the DHEA analogue is present in the composition of the invention in the form of an extract of wild yam, such as the product sold by the company Active Organics under the trade name Actigen Y.

The expression “DHEA derivatives” means both the biological derivatives thereof and the chemical derivatives thereof. Biological derivatives include, in particular, Δ5-androstene-3,17-diol and Δ4-androstene-3,17-dione, although this list is not limiting. Chemical derivatives include, in particular, DHEA salts, in particular water-soluble salts, such as DHEA sulfate. Other derivatives of the compound include esters, such as the hydroxycarboxylic acid esters of DHEA described, in particular, in U.S. Pat. No. 5,736,537 and other esters such as DHEA salicylate, acetate, valerate (or n-heptanoate) and enanthate. Other suitable DHEA derivatives include (DHEA carbamates, 2-hydroxymalonate esters of DHEA and amino acid esters of DHEA) described in patent application FR 00/03846 in the name of the L'Oreal. Needless to say, this list is not limiting.

The chemical and biological precursors and derivatives of DHEA are denoted hereinbelow as “DHEA analogues”.

DHEA and/or the analogues thereof may be present in an amount ranging from 0.0001% to 20% by weight, preferably from 0.001% to 5% by weight, relative to the total weight of the composition.

Glycosaminoglycans are anionic polysaccharides which are synthesized by fibroblasts, which are linked to polypeptides to form proteoglycans which form an integral part of the extracellular matrix of the dermis (connective tissue) with collagens, elastin and other glycoproteins. The main glycosaminoglycans are hyaluronic acid and chondroitin sulfates. They contribute towards the moisturization, suppleness and distribution of collagen fibers and thus participate in preventing dryness of the skin and in improving its tonicity.

The expression “compounds which increase glycosaminoglycan synthesis” means any compound which is capable of stimulating the production of glycosaminoglycans, in particular, of hyaluronic acid and chondroitin sulfates, by fibroblasts.

In the present invention, the compound which increases glycosaminoglycan synthesis is preferably present in the form of a natural extract. Examples of such natural extracts comprise certain algal or yeast extracts, in particular: (a) an extract of Padina pavonica which is a brown alga living in the shallow waters of the Mediterranean, available in particular from the company Alban Muller International under the trade name HPS3; and (b) an extract of Saccharomyces cerevisiae available in particular from the company Silab under the trade name Firmalift or from the company LSN under the trade name Cytovitin.

The natural extract containing the compound which increases glycosaminoglycan synthesis represents, for example, from 0.0001% to 20% and preferably from 0.001% to 5% of the total weight of the composition of the invention.

According to a preferred embodiment of the invention, the composition also contains at least one metalloprotease inhibitor, such as lycopene, isoflavones, derivatives thereof or plant extracts containing them, in particular extracts of soybean (sold, for example, by the company Ichimaru Pharcos under the trade name Flavosterone SB), of red clover, of flax, of kakkon or of sage. As a variant or in addition, the composition of the invention may contain an agent which stimulates the growth and/or metabolism of fibroblasts, such as plant proteins or polypeptides, extracts, in particular, of soybean, for example, an extract of soybean sold by the company LSN under the name Eleseryl SH-VEG 8 or sold by the company Silab under the trade name Raffermine, and/or an agent which promotes the differentiation and/or growth and/or metabolism of keratinocytes, such as retinol or derivatives thereof or certain plant extracts such as an extract of lupin sold by the company Silab under the trade name Structurine, and/or an agent which stimulates skin microcirculation, such as an extract of bitter orange sold by the company Silab under the trade name Remoduline.

The concentration of these optional additional agents in the composition of the invention may range from 0.0001% to 20% by weight, preferably from 0.001% to 5% by weight relative to the total weight of the composition.

The composition of the invention may also contain an agent which promotes collagen synthesis, such as ascorbic acid or derivatives thereof, an exfoliant such as α- or β-hydroxy acids, a protecting agent or UV screening agent, an agent which improves the skin barrier, such as ceramides, and/or a moisturizer such as polyols or calcium pantothenate.

According to a particularly preferred embodiment of the invention, the composition contains an extract of wild yam, an extract of Padina pavonica and at least one isoflavone-rich and/or protein-rich extract of soybean.

According to still another embodiment, the composition of the invention contains, as a variant or in addition, a protein-rich extract of lupin.

The composition of the invention is intended for topical application to the skin and consequently contains a physiologically acceptable medium, i.e. a medium which is compatible with the skin, its integuments and mucous membranes.

The composition of the invention may be in any form of presentation normally used in cosmetics and dermatology, and it may be, in particular, in the form of an optionally gelled aqueous solution, an optionally two-phase dispersion of the lotion type, an emulsion obtained by dispersing a fatty phase in an aqueous phase (O/W) or conversely (W/O), or a triple emulsion (W/O/W or O/W/O) or a vesicular dispersion of ionic and/or nonionic type. These compositions are prepared by the usual methods.

This composition may be more or less fluid and may have the appearance of a white or colored cream, an ointment, a milk, a lotion, a serum, a paste or a mousse. It may optionally be applied to the skin in the form of an aerosol. It may also be in solid form, in particular, in the form of a stick. It may be used as a care product and/or as a make-up product for the skin.

In a known manner, the composition of the invention may also contain adjuvants that are common in cosmetics, such as hydrophilic or lipophilic gelling agents, preserving agents, antioxidants, solvents, fragrances, fillers, screening agents, pigments, odor absorbers and dyestuffs. The amounts of these various adjuvants are those conventionally used in the field under consideration, and, for example, from 0.01% to 20% of the total weight of the composition. Depending on their nature, these adjuvants may be introduced into the fatty phase, into the aqueous phase, into lipid vesicles and/or into nanoparticles. In any case, these adjuvants, and the proportions thereof, will be selected so as not to harm the desired properties of the combination of active agents according to the invention.

When the composition of the invention is an emulsion, the proportion of the fatty phase may range from 2% to 80% by weight, preferably from 3% to 50% by weight relative to the total weight of the composition. The oils, emulsifiers and co-emulsifiers used in the composition in emulsion form are selected from those used conventionally in the field under consideration. The emulsifier and the co-emulsifier are present in the composition in an amount ranging from 0.3% to 30% by weight, preferably from 0.5% to 20% by weight relative to the total weight of the composition.

Suitable oils which may be used in the invention include mineral oils (liquid petroleum jelly), oils of plant origin (avocado oil, soybean oil), oils of animal origin (lanolin), synthetic oils (perhydrosqualene), silicone oils (cyclomethicone) and fluoro oils (perfluoropolyethers). Fatty substances which may also be used include fatty alcohols (cetyl alcohol), fatty acids, waxes (carnauba wax, ozokerite) and butters (karite butter).

Suitable emulsifiers and co-emulsifiers which may be used in the invention, include, for example, fatty acid esters of polyethylene glycol, such as PEG-20 stearate, and fatty acid esters of glycerol, such as glyceryl stearate.

Suitable hydrophilic gelling agents include, in particular, carboxyvinyl polymers (carbomer), acrylic copolymers such as acrylate/alkylacrylate copolymers, polyacrylamides, polysaccharides, natural gums and clays, and, as lipophilic gelling agents, modified clays such as bentones, metal salts of fatty acids, hydrophobic silica and polyethylenes.

The composition of the invention may be used for cosmetic purposes, to prevent or treat the signs of ageing of the skin, in particular the loss of suppleness and/or the dryness of the skin and/or slackening of the skin.

An aspect of the present invention is thus also a cosmetic process which prevents and/or treats the signs of ageing of the skin, comprising the topical administration of the composition described above.

Having now generally described this invention, a further understanding can be obtained by reference to certain specific examples which are provided herein for purposes of illustration only and are not intended to be limiting unless otherwise specified. In these examples, the amounts are indicated as percentages by weight.

### EXAMPLE 1

### Emulsion in Cream Form

| | |
|---|---|
| Mixture of cetearyl alcohol and of cetearyl glucoside | 4% |
| Mixture of glyceryl stearate and PEG-100 stearate | 1% |
| Cetyl alcohol | 0.5% |
| C₁₂₋₁₅ alkyl benzoate | 2% |
| Hydrogenated polyisobutene | 5% |
| Petroleum jelly | 2% |
| Plant oils | 3.5% |
| Silicone wax | 2% |
| Volatile silicone | 5% |
| Extract of Padina pavonica | 1% |
| Extract of wild yam | 0.7% |
| Isoflavone-rich extract of soybean | 1% |
| PEG-20 | 1% |
| Alcohol | 3% |
| Glycerol | 7% |
| Gelling agents | 1.5% |
| UVA and UVB screening agents | 12.5% |
| Neutralizing agent | 1.5% |
| Preserving agents | qs |
| Water | qs 100% |

### EXAMPLE 2

### Emulsion Is Fluid Form

| | |
|---|---|
| Mixture of glyceryl stearate and PEG-100 stearate | 3% |
| Cetyl alcohol | 0.5% |
| Stearic acid | 0.6% |
| Caprylic and capric triglycerides | 2% |
| C₁₂₋₁₅ alkyl benzoate | 2% |
| Octyldodecanol | 0.5% |
| Extract of Saccharomyces cerevisiae | 1% |
| Extract of wild yam | 0.5% |
| Protein-rich extract of soybean | 1% |
| Alcohol | 3% |
| Glycerol | 5% |
| Volatile silicone | 5.5% |
| Gelling agents | 4.3% |
| UVA and UVB screening agents | 12.5% |
| Neutralizing agent | 1.4% |
| Silica microbeads | 2% |
| Preserving agents | qs |
| Water | qs 100% |

### EXAMPLE 3

### Anti-ageing Serum

| | |
|---|---|
| Pentaerythritol tetraoctanoate | 1.4% |
| Sodium hydroxide | 0.7% |
| Citric acid | 1.2% |
| Gelling agents | 2.5% |
| Silicone oil | 3% |
| Extract of Padina pavonica | 1% |
| Extract of wild yam | 1% |
| Protein-rich extract of soybean | 5% |
| Isoflavone-rich extract of soybean | 1% |
| Extract of sage leaves | 0.02% |
| Alcohol | 5% |
| Glycerol | 3% |
| Hexylene glycol | 0.6% |
| Preserving agents | qs |
| Water | qs 100% |

The disclosure of French priority Application Number 0009221 filed Jul. 13, 2000 is hereby incorporated by reference into the present application.

Obviously, numerous modifications and variations of the present invention are possible in light of the above teachings. It is, therefore, to be understood that within the scope of the appended claims, the invention may be practiced otherwise than as specifically described herein

What is claimed as new and is intended to be secured by Letters Patent is:

1. A composition, comprising:
in a physiologically acceptable medium, DHEA and/or a chemical or biological precursor or derivative thereof, in combination with at least one compound which increases glycosaminoglycan synthesis, or a natural extract containing the compound.

2. The composition according to claim 1, wherein said natural extract is selected from the group consisting of an extract of Padina pavonica and an extract of Saccharomyces cerevisiae.

3. The composition according to claim 1, which comprises from 0.0001% to 20% by weight of said natural extract relative to the total weight of the composition.

4. The composition according to claim 3, which comprises from 0.001% to 5% by weight of said natural extract relative to the total weight of the composition.

5. The composition according to claim 1, wherein said chemical precursor of DHEA is selected from the group consisting of sapogenins, derivatives thereof and plant extracts containing them.

6. The composition according to claim 5, wherein said chemical precursor of DHEA is selected from the group consisting of diosgenin, hecogenin, hecogenin acetate, smilagenin and sarsapogenin.

7. The composition according to claim 5, wherein said plant extract is selected from the group consisting of Dioscorea extracts.

8. The composition according to claim 7, wherein said Dioscorea extract is an extract of the root of wild yam.

9. The composition according to claim 1, which is topically applied to the skin.

10. The composition according to claim 1, wherein the DHEA and/or the precursor or derivative thereof is present in the composition in an amount ranging from 0.0001% to 20% by weight relative to the total weight of the composition.

11. The composition according to claim 10, wherein the DHEA and/or the precursor or derivative thereof is present in an amount ranging from 0.001% to 5% by weight relative to the total weight of the composition.

12. The composition according to claim 1, which further comprises at least one metalloprotease inhibitor, an agent which stimulates the growth and/or metabolism of fibroblasts, and/or an agent which promotes the differentiation and/or growth and/or metabolism of keratinocytes, and/or an agent which stimulates skin microcirculation.

13. The composition according to claim 12, wherein said metalloprotease inhibitor is selected from the group consisting of an isoflavone-rich extract of soybean, an extract of red clover, an extract of flax, an extract of kakkon and an extract of sage.

14. The composition according to claim 12, wherein said agent which stimulates the growth and/or metabolism of fibroblasts is a protein-rich extract of soybean.

15. The composition according to claim 14, which comprises an extract of wild yam, an extract of Padina pavonica and at least one isoflavone-rich and/or protein-rich extract of soybean.

16. The composition according to claim 1, which is in the form of a gelled aqueous solution, a two-phase dispersion, an emulsion of a dispersion of a fatty phase in an aqueous phase (O/W) or a dispersion of an aqueous phase in a fatty phase (W/O), a triple emulsion or a vescular dispersion of the ionic and/or nonionic type.

17. The composition according to claim 1, which is in the form of an emulsion in which the amount of fatty phase ranges from 2% to 80% by weight and the amount of emulsifier and co-emulsifier ranges from 0.3% to 30% by weight relative to the total weight of the composition.

18. The composition according to claim 16, wherein the oil of the fatty phase is a mineral oil, an oil of plant origin, an oil of animal origin, a synthetic oil, a silicone oil, a fluoro oil, a fatty alcohol, a wax or a butter.

19. A method of treating the signs of ageing of the skin, comprising:
applying the composition of claim 1 to the skin.

20. A method of cosmetically treating the skin, comprising:
applying the composition of claim 1 to the skin, thereby treating the signs of ageing of the skin.

21. A method of cosmetically treating the skin, comprising:
applying the composition of claim 1 to the skin, thereby treating the loss of suppleness and/or the dryness of the skin and/or slackening of the skin.
